(19) United States
(12) Patent Application Publication
(10) Pub. No.: US 2005/0118161 A1
(43) Pub. Date: Jun. 2, 2005

(54) Hevein-binding monoclonal antibodies

(76) Inventors: Marja-Leena Laukkanen, Espoo (FI); Hans Soderlund, Espoo (FI); Soili Makinen-Kiljunen, Helsinki (FI); Tari Haahtela, Helsinki (FI); Kristiina Takkinen, Espoo (FI)

Correspondence Address: BIRCH STEWART KOLASCH & BIRCH, PO BOX 747, FALLS CHURCH, VA, 22040-0747, US

(21) Appl. No.: 10/477,830

(22) PCT Filed: May 17, 2002
(86) PCT No.: PCT/FI02/00423

(30) Foreign Application Priority Data
May 18, 2001 (FI) 20011055

(51) Int. Cl. A61K 39/395; C07H 21/04; C07K 16/18; C12N 5/06

(52) U.S. Cl. 424/130.1; 435/69.1; 435/320.1; 435/326; 530/388.1; 536/23.53

(57) ABSTRACT

This invention relates to antibody engineering technology. More particularly, the present invention relates to human IgE antibodies and derivatives thereof, which bind allergenic hevein with high affinity and specificity. The present invention also relates to processes for makings and engineering such hevein-binding monoclonal antibodies and to methods for using these antibodies and derivatives thereof in the field of immunodiagnostics, enabling qualitative and quantitative determination of allergenic hevein in biological and raw material samples, as well as in immunotherapy, enabling blocking of allergenic hevein in allergic patients.

### FIELD OF THE INVENTION

This invention relates to antibody engineering technology. More particularly, the present invention relates to human IgE antibodies and derivatives thereof, which bind allergenic hevein with high affinity and specificity. The present invention also relates to processes for making and engineering such hevein-binding monoclonal antibodies and to methods for using these antibodies and derivatives thereof in the field of immunodiagnostics, enabling qualitative and quantitative determination of allergenic hevein in biological and raw material samples, as well as in immunotherapy, enabling blocking of allergenic hevein in allergic patients.

### BACKGROUND OF THE INVENTION

Almost 20% of the population world-wide are suffering from allergy. Consequently, it is a health problem of increasing seriousness. Allergy is a hypersensitivity reaction against substances in air, food or water, which are normally harmless (Corry and Kheradmand, 1999). A new and foreign external agent triggers an allergic reaction, which aims at disposal of that agent from the body. In IgE-mediated allergic reactions, also called immediate or type I hypersensitivity reactions, under the first exposure of a foreign substance, allergen, to the body, IgE-bearing B-cells begin to produce soluble IgE molecules which will then bind to high-affinity IgE receptors present on the surface of a wide variety of cells, most importantly to mast cells. If the same foreign substance is encountered again, the cross-linking of the receptor-bound IgE molecules by the allergen occurs, resulting in cellular activation followed by the release of toxic products such as histamines, which will elicit the signs and symptoms of an allergic reaction.

Latex allergy is a serious medical problem with an increasing number of patients (Slater, 1994, Turjanmaa et al., 1996). Latex is a complex intracellular product, a milky sap, produced by the laticiferous cells of the rubber tree, Hevea brasiliensis, which is used in a variety of everyday articles, e.g. for the production of gloves, balloons, and condoms, and in manufacturing of medical devices. Latex allergy is a serious problem especially with health-care workers, rubber industry workers and patients having undergone several surgical procedures. Latex allergy has also been reported to be associated with pollen allergies and food allergies (Nel and Gujuluva, 1998). The cross-reactivity between latex and food allergens is established as the latex-fruit syndrome that might be the consequence of hevein-like protein domains or similar epitopes (Brehler et al., 1997, Chen et al., 1998, Mikkola et al., 1998). Many latex proteins have been identified as allergens (Breiteneder and Scheiner, 1998). One of the major latex allergens is hevein, which is a defence protein involved in, for instance, the inhibition of several chitin-containing fungi (Lee et al., 1991, Alenius et al., 1996, Chen et al., 1997). Hevein is a small chitin-binding protein of 43 amino acids with four disulphide bonds. Its three-dimensional structure has been determined by X-ray diffraction and NMR (Rodriguez-Romero et al., 1991; Andersen et al., 1993).

IgE antibodies distinctively recognise allergenic epitopes, which would be useful in clinics or immunodiagnostics for detecting and determining allergen concentrations of complex materials. Further, allergenic epitopes are usually different from the immunogenic epitopes of proteins. This fact has hampered the production of monoclonal antibodies capable of specific binding of allergenic epitopes by conventional methodology such as hybridoma technology. It has been recently shown that the development of allergen-specific IgE antibodies is possible by the phage display technology (Steinberger et al., 1996). This methodology is giving new tools to produce allergen-specific recombinant antibodies that can be produced in consistent quality for clinical and diagnostic applications.

### SUMMARY OF THE INVENTION

We describe in this application the development and characterisation of human IgE antibody fragments that bind allergenic hevein with affinity and specificity high enough to be utilised as reagents in immunoassays designed for the qualitative and quantitative measurement of hevein in biological samples and, in immunotherapy of allergic patients. Specifically, the present invention describes selection of human IgE antibodies specific to hevein by the phage display technique, and the characterisation of the binding properties of the engineered antibody fragments produced in E.coli.

This invention thus provides new reagents to be utilised in different kinds of immunoassay protocols, as well as human immunotherapy. The invention also permits guaranteed continuous supply of these specific reagents of uniform quality, eliminating inherent batch-to-batch variation of polyclonal antisera. These advantageous effects permit the manufacture of new, specific and economical immunodiagnostic assays of uniform quality.

Consequently, one specific object of the present invention is to provide human IgE mono-clonal antibodies, fragments thereof, or other-derivatives of such antibodies, which bind hevein with affinity and specificity high enough to allow qualitative and quantitative measurement of hevein in biological samples, as well as their use in immunotherapy. The monovalent antibodies of the present invention demonstrate a specific binding to allergenic hevein.

Another object of the present invention is to provide cDNA clones encoding hevein-specific antibody chains, as well as constructs and methods for expression of such clones to produce hevein-binding antibodies, fragments thereof or other derivatives of such antibodies.

A further object of this invention is to provide methods of using such hevein-binding antibodies, fragments thereof or other derivatives of such antibodies, or combinations of them for qualitative and quantitative measurement of hevein in biological samples. Additionally, this invention provides hevein-binding antibodies, fragments thereof or other derivatives of such antibodies, or combinations of them for immunotherapy in allergic patients.

Other objects, features and advantages of the present invention will be become apparent from the following drawings and detailed description. It should be understood, however, that the detailed description and the specific examples, while indicating preferred embodiments of the invention, are given for illustration only, since various changes and modifications within the spirit and scope of the invention will become apparent to those skilled in the art from this detailed description.

### BRIEF DESCRIPTION OF THE DRAWINGS

The figures of the constructions are not in scale.

FIG. 1 shows a schematic presentation of an intact human IgE subclass antibody, Fab fragment and single-chain antibody (scFv). The antigen-binding site is indicated by a triangle.

FIG. 2 shows schematically the panning procedure.

FIG. 3 shows a schematic presentation of the scFv phage display vector used for the construction of scFv phage libraries.

FIG. 4 shows the deduced amino acid sequence of the heavy chain variable region of the 1A4 and 1C2 antibodies. The Complementarity Determining Regions (CDRs) are underlined. Numbering is according to Kabat (Kabat et al., 1991).

FIG. 5 shows the deduced amino acid sequence of the light chain variable region of the 1A4 and 1C2 antibodies. CDRs are underlined. Numbering is according to Kabat (Kabat et al., 1991).

FIG. 6a shows the curve obtained from the competitive ELISA of 1A4 Fab fragment with human IgG1 subtype whose binding to hevein has been inhibited by latex polypeptide.

FIG. 6b shows the curve obtained from the competitive ELISA of 1 C2 Fab fragment with human IgG1 subtype whose binding to hevein has been inhibited by latex polypeptide.

FIG. 7 shows the result of the competitive ELISA. The binding of 1A4 Fab fragments with human IgG1 subtype to hevein is inhibited by allergenic epitopes (6-mer and 13-mer) of the hevein.

### ABBREVIATIONS

cDNA complementary deoxyribonucleic acid
CDR complementarity determining region
DNA deoxyribonucleic acid
E. coli Escherichia coli
ELISA enzyme-linked immunosorbent assay
Fab fragment with specific antigen binding
Fd variable and first constant domain of a heavy chain
Fv variable regions of an antibody with specific antigen binding
GFP green fluorescent protein
IgE immunoglobulin E
mRNA messenger ribonucleic acid
NMR nuclear magnetic resonance
PCR polymerase chain reaction
RNA ribonucleic acid
scfv single-chain antibody
supE⁻ a genotype of bacterial strain carrying a glutamine-inserting amber suppressor tRNA
V_{H} variable region of a heavy chain
V_{L} variable region of a light chain

### DETAILED DESCRIPTION OF THE INVENTION

The following definitions are provided for some terms used in this specification. The terms, “immunoglobulin”, “heavy chain”, “light chain” and “Fab” are used in the same way as in the European Patent Application No. 0125023.

“Antibody” in its various grammatical forms is used herein as a collective noun that refers to a population of immunoglobulin molecules and/or immunologically active portions of immunoglobulin molecules, i.e., molecules that contain an antigen binding site or a paratope.

An “antigen-binding site”, a “paratope”, is the structural portion of an antibody molecule that specifically binds an antigen.

Exemplary antibodies are those portions of an immunoglobulin molecule that contain the paratope, including those portions known as Fab and Fv.

“Fab” (fragment with specific antigen binding), a portion of antibodies can be prepared by the proteolytic reaction of papain on substantially intact antibodies by methods that are well known. See for example, U.S. Pat. No. 4,342,566. Fab fragments can also be produced by recombinant methods, which are well known to those skilled in the art. See, for example, U.S. Pat. No. 4,949,778.

“Domain” is used to describe an independently folding part of a protein. General structural definitions for domain borders in natural proteins are given in Argos, 1988.

A “variable domain” or “Fv” is used to describe those regions of the immunoglobulin molecule, which are responsible for antigen or hapten binding. Usually these consist of approximately the first 100 amino acids of the N-termini of the light and the heavy chain of the immunoglobulin molecule.

“Single-chain antibody” (scFv) is used to define a molecule in which the variable domains of the heavy and light chain of an antibody are joined together via a linker peptide to form a continuous amino acid chain synthesised from a single mRNA molecule (transcript).

“Linker” or “linker peptide” is used to describe an amino acid sequence that extends between adjacent domains in a natural or engineered protein.

A “hevein-binding antibody” is an antibody, which specifically recognises hevein and binds to it, due to interaction mediated by its variable domains.

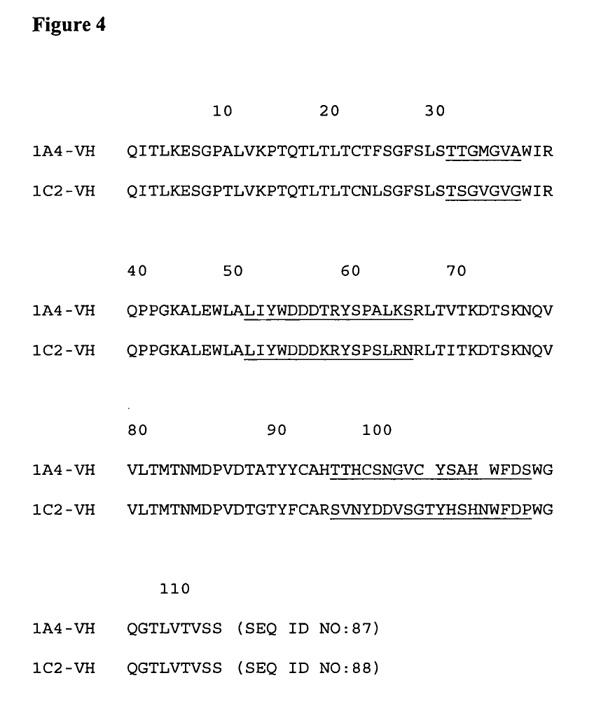

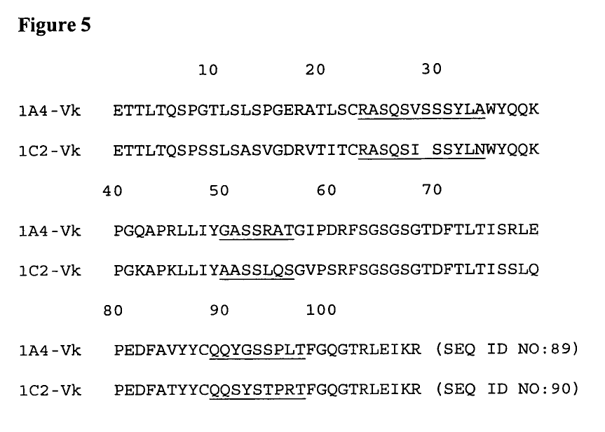

As examples of fragments of such antibodies falling within the scope of the invention we disclose here scFv fragments of 1A4 and 1C2 as shown in FIGS. 4 and 5. In one preferred embodiment, the present invention thus provides derivatives of hevein-binding antibodies, e.g. Fab fragments or scFv fragments. It will be appreciated that mutant versions of the CDR sequences or complete V_{L} and V_{H} sequences having one or more conservative substitutions which do not substantially affect binding capability, may alternatively be employed.

For use in immunoassay, e.g. for qualitative or quantitative determination of hevein in biological samples, antibodies and antibody derivatives of the invention may be labelled. For these purposes, any type of label conventionally employed for antibody labelling is acceptable.

For use in immunotherapy, e.g. for blocking allergenic hevein in allergic patients, antibodies and antibody derivatives of the invention may be labelled. For these purposes, any pharmaceutically acceptable label conventionally employed for antibody labelling is appropriate.

In another aspect, the present invention also provides DNA molecules encoding an antibody or antibody derivative of the invention, and fragments of such DNAs, which encode the CDRs of the V_{L} and/or V_{H} region. Such a DNA may be cloned in a vector, more particularly, for example, an expression vector which is capable of directing expression of antibody derivatives of the invention, or at least one antibody chain or a part of one anti-body chain.

In a further aspect of the invention, host cells are provided, selected from bacterial cells, yeast cells, fungal cells, insect cells, plant cells and mammalian cells, containing a DNA molecule of the invention, including host cells capable of expressing an antibody or antibody derivative of the invention. Thus, antibody derivatives of the invention may be prepared by culturing host cells of the invention expressing the required antibody chain(s), and either directly recovering the desired protein or, if necessary, initially recovering and combining individual chains.

The above-indicated scFv fragments were obtained by biopanning of a human IgE scFv-phage library using allergenic recombinant hevein. The human IgE scFv-phage library was constructed from mRNAs isolated from lymphocytes of a latex-allergic patient. The variable region of the light and heavy chain cDNAs were synthesised using human IgE-specific primers for Fd cDNAs and human kappa (κ) and lambda (λ) light chains using human κ and λ chain specific primers. The variable regions of the light and heavy chains were amplified by PCR using human κ and λ chain specific primers for Vκ and Vλ cDNAs and human IgE specific primers for V_{H} cDNAs, respectively. The human IgE scFv library was constructed by cloning the variable region cDNAs into a scFv phage display vector using restriction sites introduced into the PCR primers.

The human IgE scFv library was selected by phage display using a panning procedure. The human IgE scFv phage library was screened by a biotinylated allergenic recombinant hevein in solution and the binders were captured on streptavidin. The elution of phages was done with 100 mM HCl .(PH 2.2) followed by immediate neutralisation with 2 M Tris solution. The phage eluate was amplified in E. coli cells. After 5 rounds of biospanning, soluble scFv fragments were produced from isolated phages. The binding specificity of the selected scFv fragments was analysed by ELISA. Several hevein-specific scFv fragment clones were obtained.

As described herein, the phage display technique is an efficient and feasible approach to develop human IgE recombinant anti-hevein antibodies for diagnostic and therapeutic applications.

While one successful selection strategy for obtaining antibody fragments of the invention has been described, numerous variations, by which antibody fragments of the invention may be obtained, will be apparent to those skilled in the art. It may prove possible to select scFv fragments of the invention directly from a phage or microbial display library of scFv fragment or its derivatives. A phage or microbial cell, which presents a scFv fragment or other antibody fragment of the invention as a fusion protein with a surface protein, represents a still further aspect of the invention.

While microbial expression of antibodies and antibody derivatives of the invention offers means for efficient and economical production of highly specific reagents of uniform quality suitable for use in immunodiagnostic assays and immunotherapy, alternatively it may prove possible to produce such a reagent, or at least a portion thereof, synthetically. By applying conventional genetic engineering techniques, initially obtained antibody fragments of the invention may be altered, e.g. new sequences linked, without substantially altering the binding characteristics. Such techniques may be employed to produce novel hevein-binding hybrid proteins, which retain both affinity and specificity for hevein as defined hereinbefore.

The development and characterisation of the human hevein-binding recombinant antibodies and their usefulness in immunoassays is now described in more detail in the following examples.

### EXAMPLE 1

### The Recombinant Hevein-Specific scfv Fragment by Phage Display Selection

In this example the human IgE scFv library was constructed and selected by allergenic hevein in order to isolate scFv fragments with affinity and specificity to hevein. Construction of human IgE scFv phage library was prepared indirectly by constructing IgE Fab-κ and Fab-λ libraries first, and then the particular library DNAs were used for PCR amplification of variable domains of heavy and light chains.

I. Construction of the Human IgE scFv Phage Libraries

100 ml of heparinised blood was obtained from a latex-allergic patient. Lymphocytes were isolated according to an Ig-Prime kit protocol (Novagen). Per 10 ml of blood 30 ml of lysis buffer (155 mM NH₄Cl, 10 mM NH₄CO₃, 0.1 mM EDTA, pH 7.4) was added and incubated on ice for 15 min with shaking occasionally. After centrifugation at 450 g for 10 mm the lymphocytes, i.e. the white blood cell pellet, were collected. The pellet was washed twice with lysis buffer and after the final centrifugation the lymphocyte pellet was resuspended in D-solution. Lymphocyte RNAs were isolated using Promega's RNAgents Total RNA Isolation kit according to the manufacturer's protocol. The first strand cDNA synthesis was carried out using Promega's Reverse Transcription system kit. For the synthesis of Fd-fragment cDNA and light chain cDNAs the primers of the constant region of the epsilon (ε) chain (Cε1 and Cε2) and the primer of the kappa (Cκ1) and lambda (Cλ1) chain were used, respectively. Primers used for the cDNA synthesis and PCR amplifications of human IgE Fd region and light chains are showed in Table I and Table II.

PCR amplifications were carried out in two steps: a primary PCR for amplifying Fd and light chains from cDNA templates and a secondary PCR for adding restriction sites to the 5′-end of the DNA fragments obtained after a primary PCR. First the Fd region was amplified by PCR using the primers specific for the variable region of the heavy chains (VH1a-VH7a) and Cε1NotI primer. Accordingly, the kappa and lambda light chains were amplified using specific primers for variable region of the light chains (Vε1a-Vκ6b and Vλ1a-Vλ10) and Cε1NotI primer, respectively. Primers for the secondary PCR were Cκ1 and Vκ/λ1 and Cε2 for the Fd region, Vκ/λ1 and Cλ1 for the kappa light chain and Vλ1A and Cκ/λ1 for the lambda light chain. The primary PCR amplification was done at the following conditions: 1 cycle of 3 min at 93° C. for denaturation, 7 cycles of 1 min at 93° C., 30 s at 63° C. and 50 s at 58° C. for annealing and 1 min at 72° C. for elongation, 23 cycles of 1 min at 93° C., 30 s at 63° C. and 1 min at 72° C. followed by 1 cycle of 10 min at 72° C. For the secondary PCR the amplification conditions were as follows: 1 cycle of 3 min at 95° C. for denaturation, 25 cycles of 1.5 min at 94° C., 1 min at 65° C. for annealing and 1.5 min at 72° C. for elongation followed by 1 cycle of 10 min at 72° C. Between the primary and the secondary PCR and after the secondary PCR tie amplified DNA fragments were purified.

The final PCR products of the different antibody fragments were pooled and digested with appropriate restriction enzymes. Digested DNA fragments, encoding IgE Fd region and κ and λ light chains, were ligated into a phagemid vector and transformed into E. coli XL-1 Blue cells to yield an Fab-κ and Fab-λ libraries of 10⁶ independent clones. To avoid possible problems on the expression of Fab fragments on a phage particle an antibody library in scFv format was constructed. Phagemid DNAs from different libraries were isolated and used as template DNAs for amplifying the variable regions of the human IgE heavy and human light chains in order to construct human IgE scFv-κ and scFv-λ libraries.

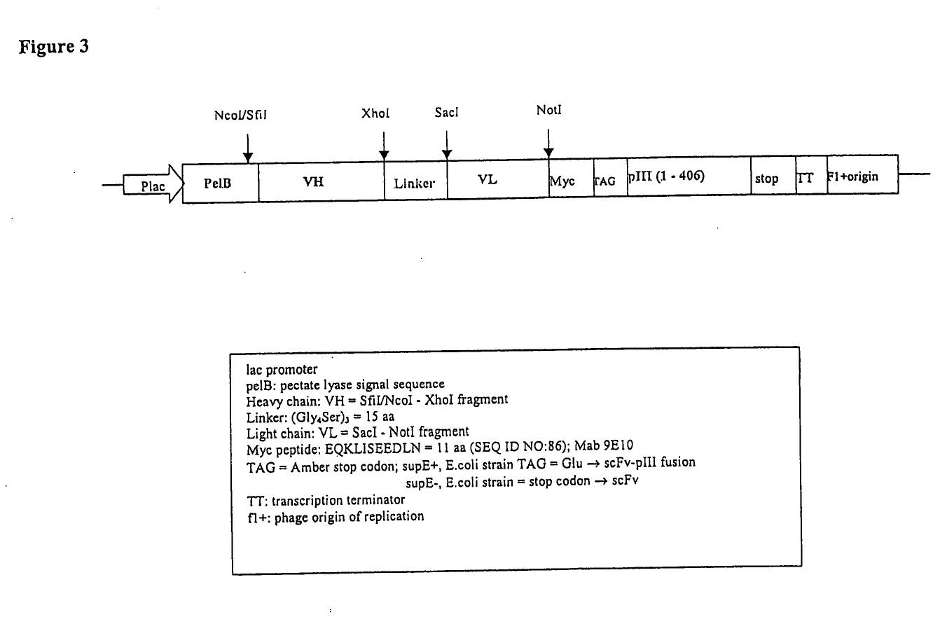

PCR amplification of the variable region of the heavy chain was carried out using human V_{H} specific primers (VH1-VH4 and VH1A). Amplification of the variable region of the light chains was done using the following primer pairs: Vκ1-Vκ7, Vκ2-Vκ8, Vκ3-Vκ9, Vκ4-Vκ10, Vκ5-Vκ11 and Vκ6-Vκ11 for human kappa chain and Vλ1-Vλ8, Vλ2-Vλ9, Vλ3-Vλ9, Vλ4-Vλ9, Vλ5-Vλ10, Vλ6-Vλ10 and Vλ7-Vλ10 for human lambda chain (see Tables III and IV). The amplified DNA fragments were purified and digested in order to ligate into a scFv phage display vector (FIG. 3). Ligation mixtures were transformed into E. coli XL-1 Blue cells resulting in the human IgE scFv-κ and scFv-λ libraries with approximately 10⁵ independent clones.

II. Selection of the Human scFv-Libraries

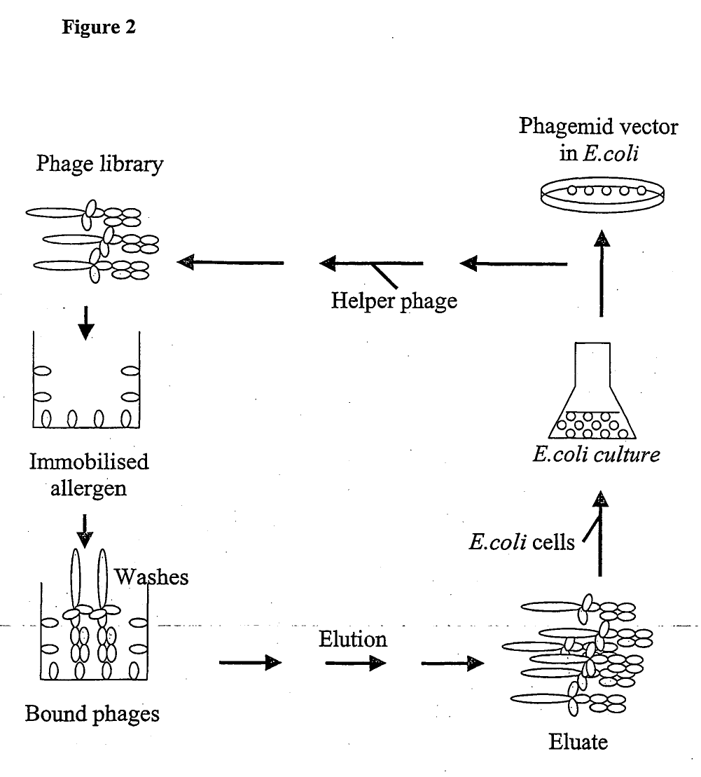

The human scFv-κ and scFv-λ libraries were selected by the phage display technique (McCafferty et al., 1990, Barbas et al., 1991). To isolate hevein-binding antibody fragments, the human IgE scFv-κ and scFv-λ libraries displayed on the surface of the bacteriophage were pooled and panned using an affinity panning procedure (FIG. 2). First the phage pools were allowed to react either with biotinylated, immunoreactive hevein or with a biotinylated control protein (background) for 1.5 h. Thereafter, the phage pools were transferred to microtitre plate wells coated with biotin binding streptavidin. After a 30-min incubation, the wells were washed 3 times with PBS and the binders were eluted with acidic buffer (100 mM HCl, pH 2.2), and immediately neutralised with 2M Tris solution. For the next panning round the eluted phage pools were amplified by infecting E. coli XL-1 Blue cells. Five rounds of panning were performed.

III. Characterisation of the Hevein-Binders

After the last panning cycle scFv phage display DNA was isolated and transformed into E. coli HB2151 (supE⁻) cells in order to express soluble scFv fragments. Between the scFv sequence and the phage gene III sequence the scFv phage display vector contains TAG-amber stop codon which will be translated as glutamate in E. coli strains with supE⁺ genotype but as a stop codon in E. coli strains with supE⁻ genotype. Sixty-two individual clones were grown in a small scale to produce soluble scFv fragments for preliminary characterisation. Clones were analysed on ELISA test using hevein-coated wells to catch the hevein-specific binders and control protein wells to see non-specific binding (data not shown). Most of the clones bound With high affinity to hevein. Nineteen of the most promising clones were sequenced (Sanger et al., 1977) and two of them were selected for further characterisation (FIGS. 4 and 5).

### EXAMPLE 2

### Cloning and Characterisation of Human Fab Fragments with Hevein-Binding Specificity

In this example the human IgE scFvs with hevein-binding specificity were converted to human Fab fragments with IgG1 subtype. Due to known difficulties in forming multimers, the 1A4 and 1C2 scFvs, obtained from the scFv antibody library, were cloned and bacterially expressed as Fab fragments (Holliger et al., 1993, Desplancq et al., 1994). The resulting antibody fragments were further characterised by a competitive ELISA.

I. Cloning of the Human Fab Fragments with Hevein-Binding Specificity

The Fd regions were amplified by overlapping PCR. The primers used for the PCR are given in Table V.

The resulting cDNAs of the Fd region and light chains were cloned into the bacterial expression vector, pKKtac and then transformed into E. coli RV308. Soluble Fab fragments designated to 1A4G and 1C2G were produced and the Fab fragments were purified by an introduced C-terminal hexahistidinyl tag on a Sepharose column with immobilised nickel to a substantial purity (data not shown).

II. Characterisation of the Human Fab Fragments

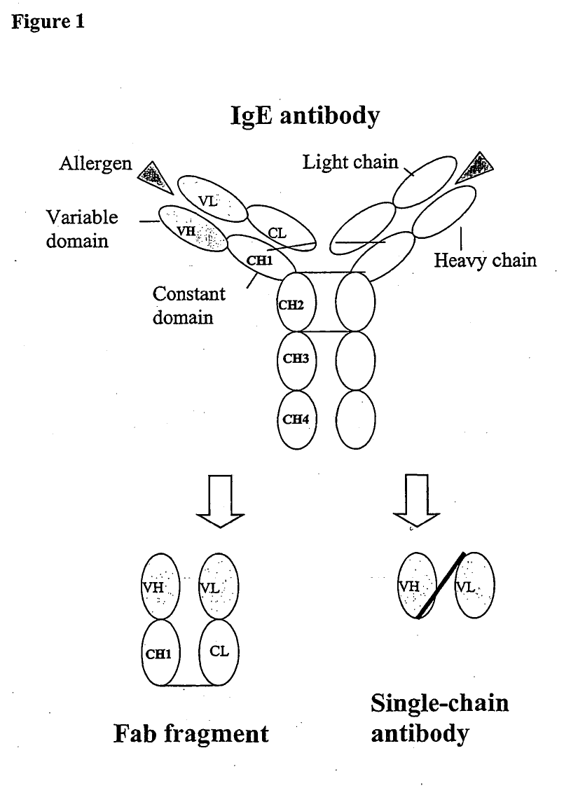

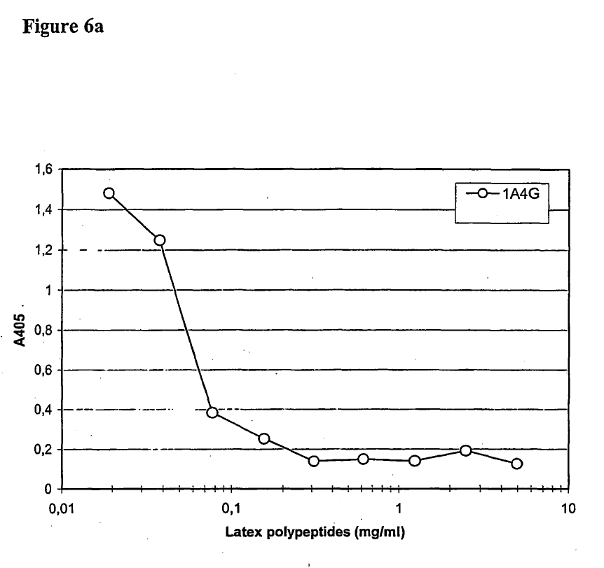

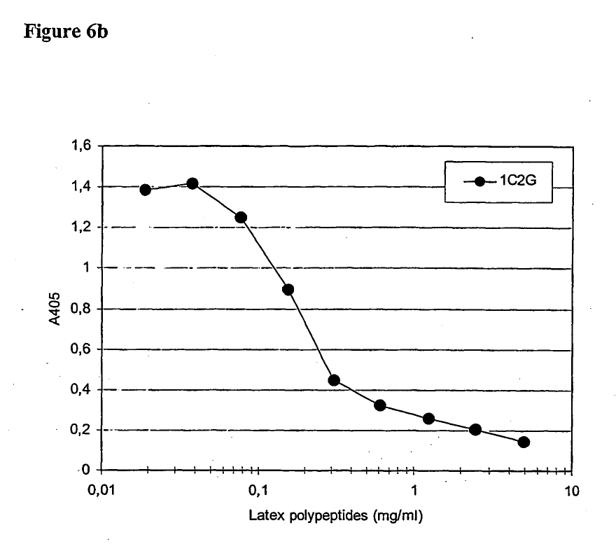

The characterisation of the purified 1A4G and 1C2G was performed by competitive ELISA. First, increasing amounts of latex polypeptides, isolated from latex examination gloves according to Alenius and co-workers (1996), were incubated with the samples, 1A4G and 1C2G, and then the reaction mixtures were applied onto microtitre plate wells coated with allergenic GFP-hevein fusion protein. Preparation of latex polypeptides have been analysed to contain high latex allergenic activity (data not shown). FIG. 6 shows the result of the competitive ELISA. The binding of the 1A4G (FIG. 6a) and 1C2G (FIG. 6b) to hevein could be inhibited by adding increasing amounts of native hevein.

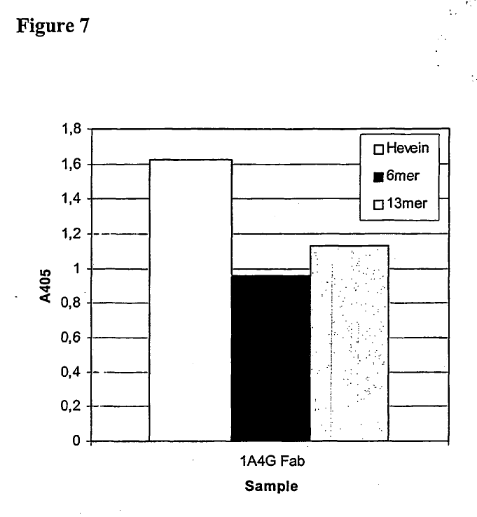

IgE antibodies bind specifically to allergenic epitopes. To study the binding specificity of the 1A4G antibody in more detail a competitive ELISA with peptides comprising the allergenic epitopes was performed (FIG. 7). Banerjee and co-workers (1997) have studied the allergenic epitopes of hevein, and they found two potential allergenic epitopes, 6-mer and 13-mer. In competitive ELISA the binding of the 1A4G to the immobilised hevein was inhibited by using the peptides of the allergenic epitopes. These results obtained in different competitive ELISAs indicate that the antibodies isolated from the antibody library can bind specifically to the recombinant hevein and the native hevein as well. In addition, the preliminary results demonstrate that the 1A4G antibody binds specifically to the allergenic epitopes of hevein.

TABLE I

Primers used for cDNA synthesis and PCR amplifica-
tion of the human IgE Fd region.

| | |
|---|---|
| Cε1: | 5′- GCTGAAGGTTTTGTTGTCGACCCAGTC -3′ |
| Cε2: | 5′- CACGGTGGGCGGGGTGAAGTCCC -3′ |
| CεNotI: | 5′- GAATGGTGCGGCCGCGCTGAAGGTTTTGTTGTCG -3′ |
| VH1a: | 5′- ATGGCCGCAGCTCAGGTKCAGCTGGTGCAG -3′ |
| VH1b: | 5′- ATGGCCGCAGCTCAGGTCCAGCTTGTGCAG -3′ |
| VH1c: | 5′- ATGGCCGCAGCTSAGGTCCAGCTGGTACAG -3′ |
| Vh1d: | 5′- ATGGCCGCAGCTCARATGCAGCTGGTGCAG -3′ |
| VH2a: | 5′- ATGGCCGCAGCTCAGATCACCTTGAAGGAG -3′ |
| VH2b: | 5′- ATGGCCGCAGCTCAGGTCACCTTGARGGAG -3′ |
| VH3a: | 5′- ATGGCCGCAGCTGARGTGCAGCTGGTGGAG -3′ |
| VH3b: | 5′- ATGGCCGCAGCTCAGGTGCAGCTGGTGGAG -3′ |
| VH3c: | 5′- ATGGCCGCAGCTGAGGTGCAGCTGTTGGAG -3′ |
| VH4a: | 5′- ATGGCCGCAGCTCAGSTGCAGCTGCAGGAG -3′ |
| VH4b: | 5′- ATGGCCGCAGCTCAGGTGCAGCTACAGCAG -3′ |
| VH5a: | 5′- ATGGCCGCAGCTGARGTGCAGCTGGTGCAG -3′ |
| VH6a: | 5′- ATGGCCGCAGCTCAGGTACAGCTGCAGCAG -3′ |
| VH7a: | 5′- ATGGCCGCAGCTCAGGTSCAGCTGGTGCAA -3′ |
| VH1A: | 5′- TTACTCGCGGCCCAGCCGGCCATGGCCGCAGCT -3′ |

TABLE II

Primers used for cDNA synthesis and PCR amplifica-
tion of human kappa and lambda chains.

Cκ1:
5′- AGGTAGGGCGCGCCTTAACACTCTCCCCTGTTGAAGC -3′
Vκ1a:
5′- ATGGCAGCGGCTRACATCCAGATGACCCAG -3′
Vκ1b:
5′- ATGGCAGCGGCTGMCATCCAGTTGACCCAG -3′
Vκ1c:
5′- ATGGCAGCGGCTGCCATCCRGATGACCCAG -3′
Vκ1d:
5′- ATGGCAGCGGCTGTCATCTGGATGACCCAG -3′
Vκ2a:
5′- ATGGCAGCGGCTGATATTGTGATGACCCAG -3′
Vκ2b:
5′- ATGGCAGCGGCTGATRTTGTGATGACTCAG -3′
Vκ3a:
5′- ATGGCAGCGGCTGAAATTGTGTTGACRCAG -3′
Vκ3b:
5′- ATGGCAGCGGCTGAAATAGTGATGACGCAG -3′
Vκ3c:
5′- ATGGCAGCGGCTGAAATTGTAATGACACAG -3′
Vκ4a:
5′- ATGGCAGCGGCTGACATCGTGATGACCCAG -3′
Vκ5a:
5′- ATGGCAGCGGCTGAAACGACACTCACGCAG -3′
Vκ6a:
5′- ATGGCAGCGGCTGAAATTGTGCTGACTCAG -3′
Vκ6b:
5′- ATGGCAGCGGCTGATGTTGTGATGACACAG -3′
Vκ/λ1:
5′- TTGTTATTGCTAGCTGCACAACCAGCAATGGCAGCGGCT -3′
Cλ1:
5′- AGGTAGGGCGCGCCTTATGAACATTCYGYAGGGGC -3′
Vλ1a:
5′- ATGGCAGCGGCTCAGTCTGTGCTGACTCAG -3′
Vλ1b:
5′- ATGGCAGCGGCTCAGTCTGTGYTGACGCAG -3′
Vλ1c:
5′- ATGGCAGCGGCTCAGTCTGTCGTGACGCAG -3′
Vλ2:
5′- ATGGCAGCGGCTCAGTCTGCCCTGACTCAG -3′
Vλ3a:
5′- ATGGCAGCGGCTTCCTATGWGCTGACTCAG -3′
Vλ3b:
5′- ATGGCAGCGGCTTCCTATGAGCTGACACAG -3′
Vλ3c:
5′- ATGGCAGCGGCTTCTTCTGAGCTGACTCAG -3′
Vλ3d:
5′- ATGGCAGCGGCTTCCTATGAGCTGATGCAG -3′
Vλ4:
5′- ATGGCAGCGGCTCAGCYTGTGCTGACTCAA -3′
Vλ5:
5′- ATGGCAGCGGCTCAGSCTGTGCTGACTCAG -3′
Vλ6:
5′- ATGGCAGCGGCTAATTTTATGCTGACTCAG -3′
Vλ7:
5′- ATGGCAGCGGCTCAGRCTGTGGTGACTCAG -3′
Vλ8:
5′- ATGGCAGCGGCTCAGACTGTGGTGACCCAG -3′
Vλ4/9:
5′- ATGGCAGCGGCTCWGCCTGTGCTGACTCAG -3′
Vλ10:
5′- ATGGCAGCGGCTCAGGCAGGGCTGACTCAG -3′

TABLE III

Primers used for PCR amplification of the human
variable regions of the heavy chain.

| | |
|---|---|
| VH1: | 5′- ATTTACTCGAGTGAGGAGACGGTGACCAGGGTGCC -3′ |
| VH2: | 5′- ATTTACTCGAGTGAAGAGACGGTGACCATTGTCCC -3′ |
| VH3: | 5′- ATTTACTCGAGTGAGGAGACGGTGACCAGGGTTCC -3′ |
| VH4: | 5′- ATTTACTCGAGTGAGGAGACGGTGACCGTGGTCCC -3′ |
| VH1A: | 5′- TTACTCGCGGCCCAGCCGGCCATGGCCGCAGCT -3′ |

TABLE IV

Primers used for PCR amplification of the human
variable regions of the light chains.

Vκ1:
5′- TTATAGAGCTCGACATCCAGATGACCCAGTCTCC -3′
Vκ2:
5′- TTATAGAGCTCGATGTTGTGATGACTCAGTCTCC -3′
Vκ3:
5′- TTATAGAGCTCGAAATTGTGTTGACGCAGTCTCC -3′
Vκ4:
5′- TTATAGAGCTCGACATCGTGATGACCCAGTCTCC -3′
Vκ5:
5′- TTATAGAGCTCGAAACGACACTCACGCAGTCTCC -3′
Vκ6:
5′- TTATAGAGCTCGAAATTGTGCTGACTCAGTCTCC -3′
Vκ7:
5′- TATAAGCGGCCGCACGTTTGATTTCCACCTTGGTCCC -3′
Vκ8:
5′- TATAAGCGGCCGCACGTTTGATCTCCAGCTTGGTCCC -3′
Vκ9:
5′- TATAAGCGGCCGCACGTTTGATATCCACTTTGGTCCC -3′
Vκ10:
5′- TATAAGCGGCCGCACGTTTGATCTCCACCTTGGTCCC -3′
Vκ11:
5′- TATAAGCGGCCGCACGTTTAATCTCCAGTCGTGTCCC -3′
Vλ1:
5′- ATTTAGAGCTCCAGTCTGTGTTGACGCAGCCGCC -3′
Vλ2:
5′- ATTTAGAGCTCCAGTCTGCCCTGACTCAGCCTGC -3′
Vλ3:
5′- ATTTAGAGCTCTCCTATGTGCTGACTCAGCCACC -3′
Vλ4:
5′- ATTTAGAGCTCTCTTCTGAGCTGACTCAGGACCC -3′
Vλ5:
5′- ATTTAGAGCTCCACGTTATACTGACTCAACCGCC -3′
Vλ6:
5′- ATTTAGAGCTCCAGGCTGTGCTCACTCAGCCGTC -3′
Vλ7:
5′- ATTTAGAGCTCAATTTTATGCTGACTCAGCCCCA -3′
Vλ8:
5′- ATATTGCGGCCGCACCTAGGACGGTGACCTTGGTCCC -3′
Vλ9:
5′- ATATTGCGGCCGCACCTAGGACGGTCAGCTTGGTCCC -3′
Vλ10:
5′- ATATTGCGGCCGCACCTAAAACGGTGAGCTGGGTCCC -3′

TABLE V

Primers used for PCR amplification of the human Fd
regions with IgE and IgG1 subtype.

5′Cε:
5′-GCTCACCGTCTCCTCAGCCTCCACACAGAGCCCATCCG-3′
3′Cε:
5′-
GCATTGCATTGCGGCCGCTTAATGGTGATGGTGATGATGGCTGAAGGT
TTTGTTGTCGACCC-3′
5′Cγ:
5′-GGTCACCGTCTCCTCAGCCTCCACCAAGGGCCC-3′
3′Cγ:
5′-
TTTAGTTTATGCGGCCGCTTAATGGTGATGATGATGGTGACAAGATTTG
GGCTCTGC-3′
5′Vε:
5′-TTACTCGCGGCCCAGCCGGCCATGGCCGCAGCT-3′
3′Vε:
5′-TGAGGAGACGGTGACC-3′
5′Cκ:
5′-GGGACACGACTGGAGATTAAAACTGTGGCTGCACCATCTGTC-3′
3′Cκ:
5′-AGGTAGGGCGCGCCTTAACACTCTCCCCTGTTGAAGC-3′
5′Vκ:
5′-ATGGCAGCGGCTGAAACGACACTCACGCAG-3′
and
5′-TTGTTATTGCTAGCTGCACAACCAGCAATGGCAGCGGCT-3′
3′Vκ:
5′-TTTAATCTCCAGTCGTGTCCC-3′.

References
Alenius, H., Kalkkinen, N., Reunala, T., Turjanmaa, K., and Palosuo, T. (1996) J. Immunol. 156, 1618-1625.
Andersen, N. H., Cao, B., Rodriguez-Romero, A., and Arreguin, B. (1993) Biochemisty 32, 1407-1422.
Argos, P. (1988) Protein Engineering, 2, 101-113.
Banerjee, B., Wang, X., Kelly, K. J., Fink, J. N., Sussman, G. L., and Kurup, V. P. (1997) J. Immunol. 159, 5724-5732.
Barbas III, C. F., Kang, A. S., Lerner, R. A., and Benkovic, S. J. (1991) Proc. Natl. Acad. Sci. U.S.A. 88, 7978-7982.
Brehler, R., Theissen, U., Mohr, C., and Luger, T. (1997) Allergy 52,404-410.
Breiteneder, H, and Scheiner, O. (1998) Int. Arch. Allergy Immunol. 116, 83-92.
Chen, Z., Posch, A., Lohaus, C., Raulf-Heimsoth, M., Meyer, H. E., and Baur, X. (1997) . J. Allergy Clin. Immunol. 99,402-409.
Chen, Z., Posch, A., Cremer, R., Raulf-Heimsoth, M., and Baur, X. (1998) J. Allergy Clin. Immunol. 102, 476-481.
Corry, D. B., and Kheradmand, F. (1999) Nature 402, B18-B23.
Desplancq, D., King, D. J., Lawson, A. D. G., and Mountain, A. (1994) Protein Eng. 7, 1027-1033.
Holliger, P., Prospero, T., and Winter, G. (1993) Proc. Natl. Acad. Sci. U.S.A. 90, 6444-6448.
Kabat, E. A., Wu, T. T., Reid-Miller, M., Perry, H. M., and Gottesman, K. S. (1991) Seguences of Proteins of Immunological Interest, 4th Ed., U.S. Dept. of Health and Human Services, Bethesda, Md.
Lee, H-i, Broekaert, W. F., and Raikhel, N. V. (1991) J. Biol. Chem. 266, 15944-15948.
McCafferty, J., Griffiths, A. D., Winter, G., and Chiswell, F. J. (1990) Nature 348, 552-554.
Mikkola, J. H., Alenius, H., Kalkkinen, N., Turjanmaa, K., Palosuo, T., and Reunala, T. (1998) J. Allergy Clin. Immunol. 102, 1005-1012.
Nel, A, and Gujuluva, C. (1998) Ann. Allergy Asthma Immunol. 81, 388-398.
Rodriguez-Romero, A., Ravichandran, K. G., and Soriano-Garcia, M. (1991) FEBS Lett. 291, 307-309.
Sanger, F., Nicklen, S., and Coulson, A. R. (1977) Proc. Natl. Acad. Sci. U.S.A. 74, 5463-5467.
Slater, J. E. (1994) J. Allergy Clin. Immunol. 94, 139-149.
Steinberger, P., Kraft, D., and Valenta, R. (1996) J. Biol. Chem. 271, 10967-10972.
Tuijanmaa, K., Alenius, H., Mäkinen-Kiljunen, S., Reunala, T, and Palosuo, T. (1996) Allergy 51, 593-602.

1. A monoclonal antibody belonging to an IgE subclass and having binding specificity to allergenic hevein, or a functional fragment or derivative thereof.

2. The monoclonal antibody according to claim 1, wherein the fragment is a scFv fragment or a Fab fragment.

3. The monoclonal antibody according to claim 2, wherein the scFv fragment is 1A4 or 1C2.

4. An isolated DNA molecule encoding the monoclonal antibody or a fragment or derivative thereof according to any one of the preceding claims, and fragments of such DNA, which encode at least one antibody chain of said antibody or antibody derivative.

5. The isolated DNA molecule according to claim 4, wherein the antibody chain is the Complementarity Determining Region (CDR) of the V_{L} and/or V_{H} region.

6. The isolated DNA molecule according to claim 4 cloned into a vector.

7. The isolated DNA molecule according to claim 6, wherein said vector is an expression vector capable of expressing antibodies, as well as fragments and derivatives thereof as claimed in any one of claims 1 to 3.

8. A host cell containing a DNA according to claim 4.

9. The host cell according to claim 8, capable of expressing a monoclonal antibody or a fragment or derivative thereof as claimed in any one of claims 1 to 3 or at least one antibody chain of said antibody or antibody derivative.

10. The host cell according to claim 9, wherein the antibody chain is the scFv fragment as claimed in claim 2 or 3.

11. A method of preparing a monoclonal antibody or a fragment or derivative thereof according to any one of claims 1 to 3, comprising the steps of
culturing a host cell according to claim 8 capable of expressing at least one of the required antibody chains, and
recovering said antibody or antibody fragment or derivative.

12. The method according to claim 11, further comprising the steps of
combining component chains after the recovery step,
introducing combined component chains into a second host cell, and
recovering said combined component chains.

13. The method according to claim 11, further comprising the step of labelling said antibody or antibody derivative.

14. A method of preparing a monoclonal antibody or a fragment or derivative thereof according to any one of claims 1 to 3, comprising the step of
synthetically producing at least a portion of said antibody or antibody derivative.

15. A phage or microbial cell, which presents an antibody fragment according to claim 2 as a fusion protein with a surface protein.

16. A method of selecting an antibody fragment according to claim 2 or 3, comprising the steps selecting said antibody fragment from a display library of antibody fragments containing a phage or cell according to claim 15.

17. A method of assaying hevein in a sample, comprising the steps of
obtaining said sample, and
assaying for hevein by employing a monoclonal antibody or a fragment or derivative thereof according to any one of claims 1 to 3.

18. A test kit comprising an antibody or a fragment or derivative thereof according to any one of claims 1 to 3 in a suitable container for transport and storage.

19. A monoclonal antibody or a fragment or derivative thereof according to any one of claims 1 to 3 for use in immunodiagnostics.

20. A monoclonal antibody or a fragment or derivative thereof according to any one of claims 1 to 3 for use in immunotherapy.


SEQUENCE LISTING (91 sequences)

SEQ ID NO: 1 - DNA, 27 bp, Artificial Sequence
  Description of Artificial Sequence oligonucleotide primer
gctgaaggttttgttgtcgacccagtc

SEQ ID NO: 2 - DNA, 23 bp, Artificial Sequence
  Description of Artificial Sequence oligonucleotide primer
cacggtgggcggggtgaagtccc

SEQ ID NO: 3 - DNA, 30 bp, Artificial Sequence
  Description of Artificial Sequence oligonucleotide primer
atggccgcagctcaggtkcagctggtgcag

SEQ ID NO: 4 - DNA, 30 bp, Artificial Sequence
  Description of Artificial Sequence oligonucleotide primer
atggccgcagctcaggtccagcttgtgcag

SEQ ID NO: 5 - DNA, 30 bp, Artificial Sequence
  Description of Artificial Sequence oligonucleotide primer
atggccgcagctsaggtccagctggtacag

SEQ ID NO: 6 - DNA, 30 bp, Artificial Sequence
  Description of Artificial Sequence oligonucleotide primer
atggccgcagctcaratgcagctggtgcag

SEQ ID NO: 7 - DNA, 30 bp, Artificial Sequence
  Description of Artificial Sequence oligonucleotide primer
atggccgcagctcagatcaccttgaaggag

SEQ ID NO: 8 - DNA, 30 bp, Artificial Sequence
  Description of Artificial Sequence oligonucleotide primer
atggccgcagctcaggtcaccttgarggag

SEQ ID NO: 9 - DNA, 30 bp, Artificial Sequence
  Description of Artificial Sequence oligonucleotide primer
atggccgcagctgargtgcagctggtggag

SEQ ID NO: 10 - DNA, 30 bp, Artificial Sequence
  Description of Artificial Sequence oligonucleotide primer
atggccgcagctcaggtgcagctggtggag

SEQ ID NO: 11 - DNA, 30 bp, Artificial Sequence
  Description of Artificial Sequence oligonucleotide primer
atggccgcagctgaggtgcagctgttggag

SEQ ID NO: 12 - DNA, 30 bp, Artificial Sequence
  Description of Artificial Sequence oligonucleotide primer
atggccgcagctcagstgcagctgcaggag

SEQ ID NO: 13 - DNA, 30 bp, Artificial Sequence
  Description of Artificial Sequence oligonucleotide primer
atggccgcagctcaggtgcagctacagcag

SEQ ID NO: 14 - DNA, 30 bp, Artificial Sequence
  Description of Artificial Sequence oligonucleotide primer
atggccgcagctgargtgcagctggtgcag

SEQ ID NO: 15 - DNA, 30 bp, Artificial Sequence
  Description of Artificial Sequence oligonucleotide primer
atggccgcagctcaggtacagctgcagcag

SEQ ID NO: 16 - DNA, 30 bp, Artificial Sequence
  Description of Artificial Sequence oligonucleotide primer
atggccgcagctcaggtscagctggtgcaa

SEQ ID NO: 17 - DNA, 33 bp, Artificial Sequence
  Description of Artificial Sequence oligonucleotide primer
ttactcgcggcccagccggccatggccgcagct

SEQ ID NO: 18 - DNA, 37 bp, Artificial Sequence
  Description of Artificial Sequence oligonucleotide primer
aggtagggcgcgccttaacactctcccctgttgaagc

SEQ ID NO: 19 - DNA, 30 bp, Artificial Sequence
  Description of Artificial Sequence oligonucleotide primer
atggcagcggctracatccagatgacccag

SEQ ID NO: 20 - DNA, 30 bp, Artificial Sequence
  Description of Artificial Sequence oligonucleotide primer
atggcagcggctgmcatccagttgacccag

SEQ ID NO: 21 - DNA, 30 bp, Artificial Sequence
  Description of Artificial Sequence oligonucleotide primer
atggcagcggctgccatccrgatgacccag

SEQ ID NO: 22 - DNA, 30 bp, Artificial Sequence
  Description of Artificial Sequence oligonucleotide primer
atggcagcggctgtcatctggatgacccag

SEQ ID NO: 23 - DNA, 30 bp, Artificial Sequence
  Description of Artificial Sequence oligonucleotide primer
atggcagcggctgatattgtgatgacccag

SEQ ID NO: 24 - DNA, 30 bp, Artificial Sequence
  Description of Artificial Sequence oligonucleotide primer
atggcagcggctgatrttgtgatgactcag

SEQ ID NO: 25 - DNA, 30 bp, Artificial Sequence
  Description of Artificial Sequence oligonucleotide primer
atggcagcggctgaaattgtgttgacrcag

SEQ ID NO: 26 - DNA, 30 bp, Artificial Sequence
  Description of Artificial Sequence oligonucleotide primer
atggcagcggctgaaatagtgatgacgcag

SEQ ID NO: 27 - DNA, 30 bp, Artificial Sequence
  Description of Artificial Sequence oligonucleotide primer
atggcagcggctgaaattgtaatgacacag

SEQ ID NO: 28 - DNA, 30 bp, Artificial Sequence
  Description of Artificial Sequence oligonucleotide primer
atggcagcggctgacatcgtgatgacccag

SEQ ID NO: 29 - DNA, 30 bp, Artificial Sequence
  Description of Artificial Sequence oligonucleotide primer
atggcagcggctgaaacgacactcacgcag

SEQ ID NO: 30 - DNA, 30 bp, Artificial Sequence
  Description of Artificial Sequence oligonucleotide primer
atggcagcggctgaaattgtgctgactcag

SEQ ID NO: 31 - DNA, 30 bp, Artificial Sequence
  Description of Artificial Sequence oligonucleotide primer
atggcagcggctgatgttgtgatgacacag

SEQ ID NO: 32 - DNA, 39 bp, Artificial Sequence
  Description of Artificial Sequence oligonucleotide primer
ttgttattgctagctgcacaaccagcaatggcagcggct

SEQ ID NO: 33 - DNA, 35 bp, Artificial Sequence
  Description of Artificial Sequence oligonucleotide primer
aggtagggcgcgccttatgaacattcygyaggggc

SEQ ID NO: 34 - DNA, 30 bp, Artificial Sequence
  Description of Artificial Sequence oligonucleotide primer
atggcagcggctcagtctgtgctgactcag

SEQ ID NO: 35 - DNA, 30 bp, Artificial Sequence
  Description of Artificial Sequence oligonucleotide primer
atggcagcggctcagtctgtgytgacgcag

SEQ ID NO: 36 - DNA, 30 bp, Artificial Sequence
  Description of Artificial Sequence oligonucleotide primer
atggcagcggctcagtctgtcgtgacgcag

SEQ ID NO: 37 - DNA, 30 bp, Artificial Sequence
  Description of Artificial Sequence oligonucleotide primer
atggcagcggctcagtctgccctgactcag

SEQ ID NO: 38 - DNA, 30 bp, Artificial Sequence
  Description of Artificial Sequence oligonucleotide primer
atggcagcggcttcctatgwgctgactcag

SEQ ID NO: 39 - DNA, 30 bp, Artificial Sequence
  Description of Artificial Sequence oligonucleotide primer
atggcagcggcttcctatgagctgacacag

SEQ ID NO: 40 - DNA, 30 bp, Artificial Sequence
  Description of Artificial Sequence oligonucleotide primer
atggcagcggcttcttctgagctgactcag

SEQ ID NO: 41 - DNA, 30 bp, Artificial Sequence
  Description of Artificial Sequence oligonucleotide primer
atggcagcggcttcctatgagctgatgcag

SEQ ID NO: 42 - DNA, 30 bp, Artificial Sequence
  Description of Artificial Sequence oligonucleotide primer
atggcagcggctcagcytgtgctgactcaa

SEQ ID NO: 43 - DNA, 30 bp, Artificial Sequence
  Description of Artificial Sequence oligonucleotide primer
atggcagcggctcagsctgtgctgactcag

SEQ ID NO: 44 - DNA, 30 bp, Artificial Sequence
  Description of Artificial Sequence oligonucleotide primer
atggcagcggctaattttatgctgactcag

SEQ ID NO: 45 - DNA, 30 bp, Artificial Sequence
  Description of Artificial Sequence oligonucleotide primer
atggcagcggctcagrctgtggtgactcag

SEQ ID NO: 46 - DNA, 30 bp, Artificial Sequence
  Description of Artificial Sequence oligonucleotide primer
atggcagcggctcagactgtggtgacccag

SEQ ID NO: 47 - DNA, 30 bp, Artificial Sequence
  Description of Artificial Sequence oligonucleotide primer
atggcagcggctcwgcctgtgctgactcag

SEQ ID NO: 48 - DNA, 30 bp, Artificial Sequence
  Description of Artificial Sequence oligonucleotide primer
atggcagcggctcaggcagggctgactcag

SEQ ID NO: 49 - DNA, 35 bp, Artificial Sequence
  Description of Artificial Sequence oligonucleotide primer
atttactcgagtgaggagacggtgaccagggtgcc

SEQ ID NO: 50 - DNA, 35 bp, Artificial Sequence
  Description of Artificial Sequence oligonucleotide primer
atttactcgagtgaagagacggtgaccattgtccc

SEQ ID NO: 51 - DNA, 35 bp, Artificial Sequence
  Description of Artificial Sequence oligonucleotide primer
atttactcgagtgaggagacggtgaccagggttcc

SEQ ID NO: 52 - DNA, 35 bp, Artificial Sequence
  Description of Artificial Sequence oligonucleotide primer
atttactcgagtgaggagacggtgaccgtggtccc

SEQ ID NO: 53 - DNA, 33 bp, Artificial Sequence; identical to SEQ ID NO: 17
  Description of Artificial Sequence oligonucleotide primer

SEQ ID NO: 54 - DNA, 34 bp, Artificial Sequence
  Description of Artificial Sequence oligonucleotide primer
ttatagagctcgacatccagatgacccagtctcc

SEQ ID NO: 55 - DNA, 34 bp, Artificial Sequence
  Description of Artificial Sequence oligonucleotide primer
ttatagagctcgatgttgtgatgactcagtctcc

SEQ ID NO: 56 - DNA, 34 bp, Artificial Sequence
  Description of Artificial Sequence oligonucleotide primer
ttatagagctcgaaattgtgttgacgcagtctcc

SEQ ID NO: 57 - DNA, 34 bp, Artificial Sequence
  Description of Artificial Sequence oligonucleotide primer
ttatagagctcgacatcgtgatgacccagtctcc

SEQ ID NO: 58 - DNA, 34 bp, Artificial Sequence
  Description of Artificial Sequence oligonucleotide primer
ttatagagctcgaaacgacactcacgcagtctcc

SEQ ID NO: 59 - DNA, 34 bp, Artificial Sequence
  Description of Artificial Sequence oligonucleotide primer
ttatagagctcgaaattgtgctgactcagtctcc

SEQ ID NO: 60 - DNA, 37 bp, Artificial Sequence
  Description of Artificial Sequence oligonucleotide primer
tataagcggccgcacgtttgatttccaccttggtccc

SEQ ID NO: 61 - DNA, 37 bp, Artificial Sequence
  Description of Artificial Sequence oligonucleotide primer
tataagcggccgcacgtttgatctccagcttggtccc

SEQ ID NO: 62 - DNA, 37 bp, Artificial Sequence
  Description of Artificial Sequence oligonucleotide primer
tataagcggccgcacgtttgatatccactttggtccc

SEQ ID NO: 63 - DNA, 37 bp, Artificial Sequence
  Description of Artificial Sequence oligonucleotide primer
tataagcggccgcacgtttgatctccaccttggtccc

SEQ ID NO: 64 - DNA, 37 bp, Artificial Sequence
  Description of Artificial Sequence oligonucleotide primer
tataagcggccgcacgtttaatctccagtcgtgtccc

SEQ ID NO: 65 - DNA, 34 bp, Artificial Sequence
  Description of Artificial Sequence oligonucleotide primer
atttagagctccagtctgtgttgacgcagccgcc

SEQ ID NO: 66 - DNA, 34 bp, Artificial Sequence
  Description of Artificial Sequence oligonucleotide primer
atttagagctccagtctgccctgactcagcctgc

SEQ ID NO: 67 - DNA, 34 bp, Artificial Sequence
  Description of Artificial Sequence oligonucleotide primer
atttagagctctcctatgtgctgactcagccacc

SEQ ID NO: 68 - DNA, 34 bp, Artificial Sequence
  Description of Artificial Sequence oligonucleotide primer
atttagagctctcttctgagctgactcaggaccc

SEQ ID NO: 69 - DNA, 34 bp, Artificial Sequence
  Description of Artificial Sequence oligonucleotide primer
atttagagctccacgttatactgactcaaccgcc

SEQ ID NO: 70 - DNA, 34 bp, Artificial Sequence
  Description of Artificial Sequence oligonucleotide primer
atttagagctccaggctgtgctcactcagccgtc

SEQ ID NO: 71 - DNA, 34 bp, Artificial Sequence
  Description of Artificial Sequence oligonucleotide primer
atttagagctcaattttatgctgactcagcccca

SEQ ID NO: 72 - DNA, 37 bp, Artificial Sequence
  Description of Artificial Sequence oligonucleotide primer
atattgcggccgcacctaggacggtgaccttggtccc

SEQ ID NO: 73 - DNA, 37 bp, Artificial Sequence
  Description of Artificial Sequence oligonucleotide primer
atattgcggccgcacctaggacggtcagcttggtccc

SEQ ID NO: 74 - DNA, 37 bp, Artificial Sequence
  Description of Artificial Sequence oligonucleotide primer
atattgcggccgcacctaaaacggtgagctgggtccc

SEQ ID NO: 75 - DNA, 38 bp, Artificial Sequence
  Description of Artificial Sequence oligonucleotide primer
gctcaccgtctcctcagcctccacacagagcccatccg

SEQ ID NO: 76 - DNA, 62 bp, Artificial Sequence
  Description of Artificial Sequence oligonucleotide primer
gcattgcattgcggccgcttaatggtgatggtgatgatggctgaaggttttgttgtcgaccc

SEQ ID NO: 77 - DNA, 33 bp, Artificial Sequence
  Description of Artificial Sequence oligonucleotide primer
ggtcaccgtctcctcagcctccaccaagggccc

SEQ ID NO: 78 - DNA, 57 bp, Artificial Sequence
  Description of Artificial Sequence oligonucleotide primer
tttagtttatgcggccgcttaatggtgatgatgatggtgacaagatttgggctctgc

SEQ ID NO: 79 - DNA, 33 bp, Artificial Sequence; identical to SEQ ID NO: 17
  Description of Artificial Sequence oligonucleotide primer

SEQ ID NO: 80 - DNA, 16 bp, Artificial Sequence
  Description of Artificial Sequence oligonucleotide primer
tgaggagacggtgacc

SEQ ID NO: 81 - DNA, 42 bp, Artificial Sequence
  Description of Artificial Sequence oligonucleotide primer
gggacacgactggagattaaaactgtggctgcaccatctgtc

SEQ ID NO: 82 - DNA, 37 bp, Artificial Sequence; identical to SEQ ID NO: 18
  Description of Artificial Sequence oligonucleotide primer

SEQ ID NO: 83 - DNA, 30 bp, Artificial Sequence; identical to SEQ ID NO: 29
  Description of Artificial Sequence oligonucleotide primer

SEQ ID NO: 84 - DNA, 39 bp, Artificial Sequence; identical to SEQ ID NO: 32
  Description of Artificial Sequence oligonucleotide primer

SEQ ID NO: 85 - DNA, 21 bp, Artificial Sequence
  Description of Artificial Sequence oligonucleotide primer
tttaatctccagtcgtgtccc

SEQ ID NO: 86 - protein, 11 aa, Myc peptide
EQKLISEEDLN

SEQ ID NO: 87 - protein, 127 aa, Homo sapiens
  SITE (31)..(37): CDR
QITLKESGPALVKPTQTLTLTCTFSGFSLSTTGMGVAWIRQPPGKALEWLALIYWDDDTRYSPALKSRLTVTKDTSKNQVVLTMTNMDPVDTATYYCAHTTHCSNGVCYSAHWFDSWGQGTLVTVSS

SEQ ID NO: 88 - protein, 129 aa, Homo sapiens
  SITE (31)..(37): CDR
QITLKESGPTLVKPTQTLTLTCNLSGFSLSTSGVGVGWIRQPPGKALEWLALIYWDDDKRYSPSLRNRLTITKDTSKNQVVLTMTNMDPVDTGTYFCARSVNYDDVSGTYHSHNWFDPWGQGTLVTVSS

SEQ ID NO: 89 - protein, 109 aa, Homo sapiens
  SITE (24)..(35): CDR
ETTLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTRLEIKR

SEQ ID NO: 90 - protein, 108 aa, Homo sapiens
  SITE (24)..(34): CDR
ETTLTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTRLEIKR

SEQ ID NO: 91 - DNA, 34 bp, Artificial Sequence
  Description of Artificial Sequence oligonucleotide primer
gaatggtgcggccgcgctgaaggttttgttgtcg